(19) United States
(12) Patent Application Publication
(10) Pub. No.: US 2008/0269771 A1
(43) Pub. Date: Oct. 30, 2008

(54) Method and apparatus for ophthalmic surgery

(76) Inventor: Samuel F.A. Fulcher, Belton, TX (US)

Correspondence Address: TROP PRUNER & HU, PC, 1616 S. VOSS ROAD, SUITE 750, HOUSTON, TX, 77057-2631, US

(21) Appl. No.: 12/150,394

(22) Filed: Apr. 28, 2008

Related U.S. Application Data
(60) Provisional application No. 60/926,278, filed on Apr. 26, 2007.

(51) Int. Cl. A61F 9/007

(52) U.S. Cl. 606/107

(57) ABSTRACT

In one embodiment of the invention, an endothelial keratoplasty device includes a distal portion with first and second holes. A vacuum is applied, via the holes, to stromal tissue of donor ophthalmic tissue. This helps the corneal surgeon insert the donor tissue into the patient's eye and couple the donor tissue to the patient's tissue, all while limiting trauma to the endothelial cells of the donor tissue. Irrigating liquid may also be administered to the patient's eye via the aforementioned holes or additional holes, ports, or voids. The irrigating fluid may remove the need for use of an anterior chamber maintaining device. Also, a protective lip located on the distal portion of the device, or elsewhere, may help protect delicate endothelial cells on the donor tissue as the donor tissue is inserted into eye.

This application claims priority to U.S. Provisional Patent Application No. 60/926,278 filed on Apr. 26, 2007 entitled METHOD AND APPARATUS FOR OPHTHALMIC SURGERY.

### BACKGROUND

Descemet's stripping automated endothelial keratoplasty or endothelial keratoplasty (EK, DSEK, DSAEK, DLEK) is a method for performing corneal transplant surgery. Endothelial keratoplasties are performed when the corneal endothelial lining has deteriorated. Unlike “open sky” or complete corneal transplants, endothelial keratoplasties entail replacing the endothelial lining of the patient to correct deteriorated corneal endothelial linings. The deteriorated lining is stripped from the inside surface of the recipient cornea through a small incision made at the edge of the cornea. The donor tissue is prepared by separating the back layers of the donor-cornea from the donor specimen, and cutting the donor cornea to size with a circular trephine. The tissue is then folded, endothelial side in, to form a so-called “taco” shape. The “taco” is grasped with forceps, and inserted into the eye through the small incision. The “taco” is encouraged to unfold, endothelial side in (or down) inside the eye using a combination air, saline, and instruments. Once unfolded, the tissue is centered in the eye and fixed into position on the inside surface of the recipient cornea with an air bubble. After a set amount of time the air bubble is removed and replaced with saline.

Problems with this technique include the folding of the tissue into the “taco”, grasping the tissue with forceps, inserting the tissue in the eye, and unfolding the tissue once inside the eye. These steps may damage the donor endothelial lining, the very lining that will replace the patient's faulty endothelial lining. This damage can lead to failure of the transplant. Furthermore, once inside the eye, the tissue may unfold upside down (endothelial side up). More manipulation is then required to flip the tissue, possibly leading to more damage to the lining.

### BRIEF DESCRIPTION OF THE DRAWINGS

The accompanying drawings, incorporated in and constituting a part of this specification, illustrate one or more implementations consistent with the principles of the invention and, together with the description of the invention, explain such implementations. The drawings are not necessarily to scale, the emphasis instead being placed upon illustrating the principles of the invention. In the drawings:

FIGS. 1a, 1b, and 1c respectively include top, bottom, and side views of one embodiment of the invention.

FIG. 2 includes a flow chart depicting a method in one embodiment of the invention.

FIG. 3 includes a side view of one embodiment of the invention.

FIG. 4 includes a side view of one embodiment of the invention.

FIG. 5 includes a side sectional view of one embodiment of the invention.

FIG. 6 includes a side sectional view of one embodiment of the invention.

FIG. 7 includes a side sectional view of one embodiment of the invention.

FIG. 8 includes a side sectional view of one embodiment of the invention.

### DETAILED DESCRIPTION

The following description refers to the accompanying drawings. Among the various drawings the same reference numbers may be used to identify the same or similar elements. While the following description provides a thorough understanding of the various aspects of the claimed invention by setting forth specific details such as particular structures, architectures, interfaces, and techniques, such details are provided for purposes of explanation and should not be viewed as limiting. Moreover, those of skill in the art will, in light of the present disclosure, appreciate that various aspects of the invention claimed may be practiced in other examples or implementations that depart from these specific details. At certain junctures in the following disclosure descriptions of well known devices, circuits, and methods have been omitted to avoid clouding the description of the present invention with unnecessary detail.

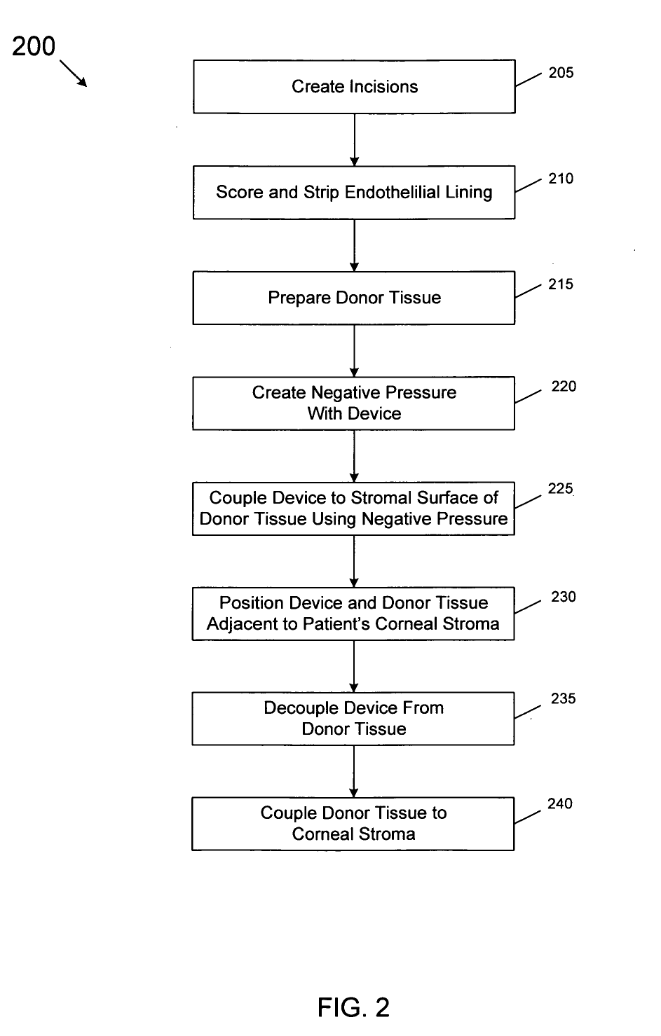

FIG. 2 discloses one embodiment of the invention which includes a method for performing endothelial keratoplasty.

In block 205, the surgeon may use a keratome to create a first incision and, in some instances, a second incision at different positions of the corneal limbus of the patient's eye. The incisions may be located respectively at temporal positions 12 o'clock and 3 o'clock of the corneal limbus. The surgeon may insert an anterior chamber maintaining device into the eye via the second incision. He or she may use the anterior chamber maintaining device to irrigate the anterior chamber with, for example, a sterile saline solution which helps maintain the shape of the eye during the surgery.

In block 210, the surgeon may insert a hook or scoring device into the eye via the first incision and subsequently score Descemet's membrane in, for example, a circular pattern. The surgeon may then strip the scored portion of the ophthalmic endothelial lining and Descemet's membrane from the corneal stroma. He or she then removes the stripped portion of the ophthalmic endothelial lining and the stripped portion of Descemet's membrane via the first incision.

In block 215, a donor cornea/sclera is received and prepared for transplant using, for example, an artificial anterior chamber. The corneal/scleral “button” may be, for example only, 16 mm in diameter. The surgeon may use a microkeratome to dissect the donor tissue leaving the ophthalmic lining, Descemet's membrane, and 100 to 200 μm of stromal tissue for transplantation into the patient. The surgeon may then use a standard cutting block and punch the donor tissue with a trephine, endothelial side up. In one embodiment of the invention, the transplant tissue button may be 8 mm to 9 mm in diameter.

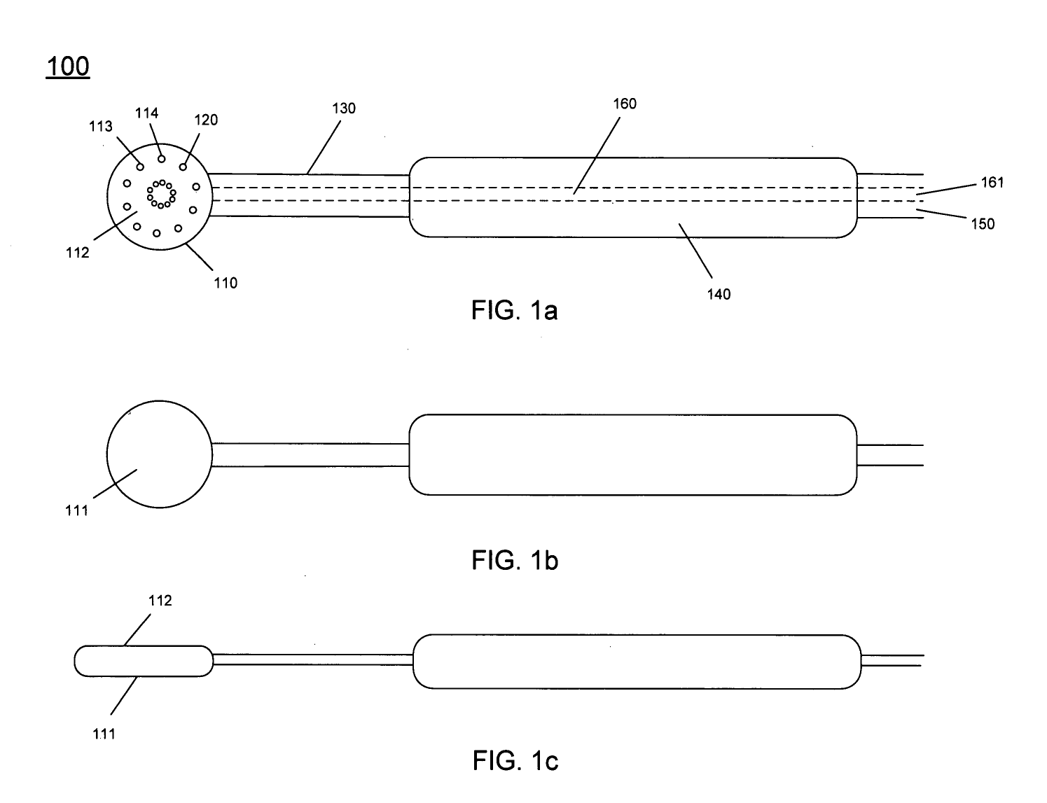

In block 220, the device illustrated in FIG. 1 may be utilized to handle the donor tissue. The device of FIG. 1 will be immediately addressed with attention then returning to the method of FIG. 2.

Accordingly, the device of FIG. 1a may include an ophthalmic surgical apparatus 100. The device may have a distal portion 110 with a first passage (not shown). The distal portion may be coupled with donor ophthalmic tissue and then inserted into the patient's eye. A proximal portion 150 may have a second passage 160 which may, in one embodiment of the invention, couple to a pressure supply (not shown). The pressure supply (e.g., peristaltic pump, syringe, etc.) may supply negative and/or positive pressure using, for example, a gas (e.g., air) and/or a liquid (e.g., sterile saline). Negative pressure may, in one embodiment of the invention, imply a mere pressure gradient relative to surrounding atmosphere inside and/or outside the eye. The device 100 may further include a conduit 140 that includes a central passage 160 to couple the passage of the distal portion (not shown) to the passage 161 of the proximal portion. The conduit 140 may be substantially collinear with the distal portion 110. The distal portion 110 may include a first surface 112 (FIG. 1a) and a second surface 111 (FIGS. 1b, 1c), the first surface 112 including a first passage 113 and a second passage 114, the first passage 113 and the second passage 114 each coupling to the passage of the distal portion, as well as passage 160.

Returning to FIG. 2 and block 220, negative pressure may be generated using the pressure supply. The negative pressure may be supplied to the device 100 via the proximal end 150 and passage 161. In block 225, the negative pressure may be supplied to the donor stromal surface of the donor tissue via passages 113, 114, 160, 161. Thus, the surgeon may manipulate the tissue in a secure manner while avoiding trauma to the endothelial cells located opposite the stromal surface.

In block 230, the surgeon may insert distal portion 110 and donor ophthalmic lining, Descemet's membrane, and stromal tissue into the patient's eye via the first incision. He or she may then manipulate the distal portion 110 to position the donor stromal surface adjacent to the patient's corneal stroma. In other words, the surgeon positions the donor stromal surface adjacent to the patient's stromal surface. This manipulation is facilitated by the device 100 being secured to the donor tissue while avoiding trauma to the donor endothelial cells.

In block 235, the surgeon may decouple the device 100 from the donor tissue by, for example, injecting a liquid (e.g., sterile saline) or gas (e.g., air) via passages 113, 114. In addition, simply discontinuing the negative pressure may also promote decoupling. Then, in block 240 he or she may couple the stromal surface of the donor endothelial lining to the patient's corneal stroma using traditional techniques such as, for example, an air bubble located adjacent to the donor endothelial cells. The air bubble is removed after a period of time such as, for example, one hour. The device 100 is then removed from the eye.

In one embodiment of the invention, the donor “button” is approximately 9 mm in diameter and the distal portion 110 of the device is substantially circular and approximately 5 mm in diameter with a 2.5 mm radius. Of course, other geometrical profiles (e.g., elliptical) are applicable as well. When negative pressure is applied to the distal portion 110 and donor tissue, the tissue may be coupled to the distal portion 110. More specifically, the distal portion may directly contact at least 19 mm² (area=Π*(radius)²=Π*(2.5 mm)²=19.63 mm²) of the stromal surface of the donor ophthalmic endothelial lining. As a result, the directly contacted portion is not folded while, for example, being inserted into the eye and placed adjacent to the patient's corneal stroma. This facilitates the proper maintenance of the donor endothelial cells.

In one instance, the entry incision to the eye may be about 5 mm in diameter. Thus, in an embodiment of the invention wherein the distal portion 110 of the device has a diameter of 5 mm, the distal portion 110 may fit in the incision. In doing so, the central portion of a tissue button having a diameter of, for example, 9 mm will be held against the distal portion 110 of the device. Thus, the central 19 mm² portion (see above example) of the donor button will not be folded and will therefore better preserve the integrity of the donor endothelial cells. The portion of the button outside of the central 19 mm² portion may experience some minor folding as the button is passed through the 5 mm incision. However, this “outer portion” should still remain relatively free of “hard” folds or creases such as those that might be encountered with a traditional “taco” fold utilized in DSAEK/DSEK procedures. In alternative embodiments of the invention, the diameter of the incision and distal portion 110 may be increased to thereby increase the size of the portion (e.g., central portion) that is held against the device and consequently unfolded.

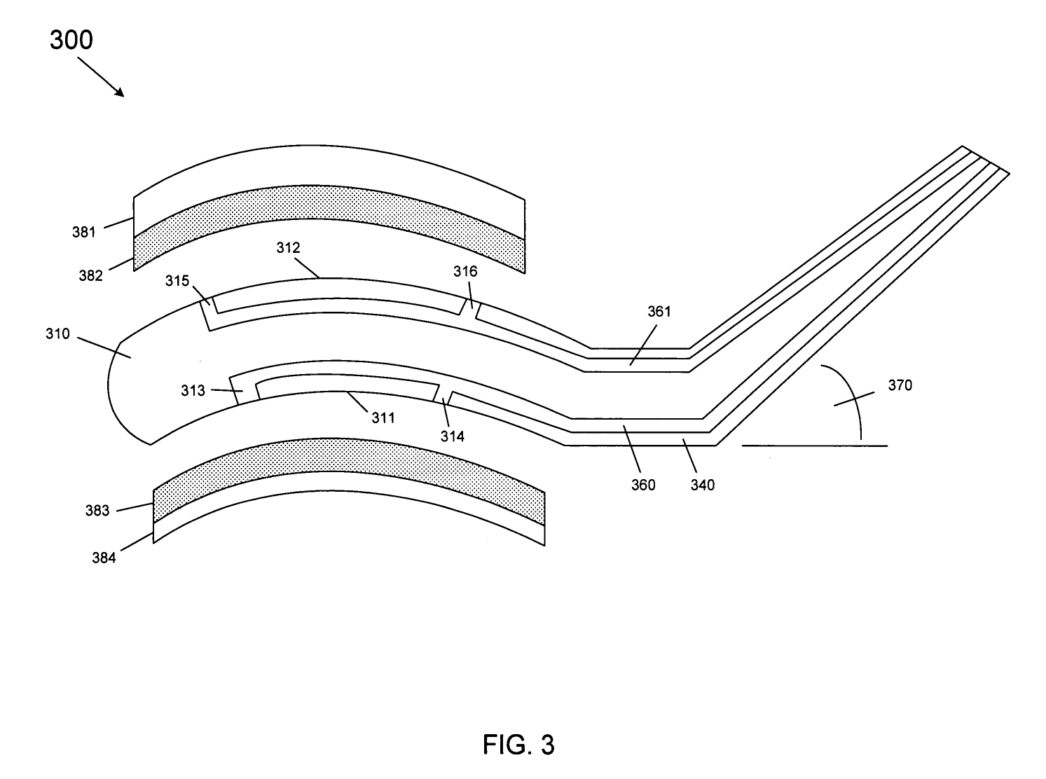

In one embodiment of the invention shown in FIG. 3, the device 300 may include a first passage 360 and a second passage 361. The second passage 361 may lie adjacent and substantially parallel to the first passage 360. In one embodiment of the invention, the passages 360, 361 may be internal or external to the device 300. The passages may share a common wall. There may be three or more passages included in certain embodiments of the invention. Returning to the example illustrated in FIG. 3, the first passage 360 may communicate with passages (i.e., holes, voids) 313, 314 to supply, for example, air pressure (e.g., positive or negative). The second passage 361 may communicate with passages 315, 316 to supply, for example, a liquid (e.g., sterile saline). The injected liquid may help the eye maintain its shape and thereby facilitate the surgery. The injected fluid may make use of the anterior chamber maintaining device unnecessary, as well as the incision associated with the anterior chamber maintaining device.

FIG. 3 illustrates the device having been recently decoupled from the donor stroma 383 and endothelial lining 384. The patient's stroma 382 and corneal epithelium 381 may receive irrigation fluid from passages 315, 316. The conduit 340 may include a bend whereby a portion of the conduit 340 is not collinear with the distal portion 310. The angle of inclination 370 may be 15 to 20 degrees but may of course vary in other embodiments of the invention. Surface 311 may include a concave portion to contour with the natural shape of the donor tissue. Surface 312 may include a convex portion to contour with the natural shape of the patient's tissue. Passages 360, 361 may each conduct different fluids in different phases (e.g., gas, liquid). Passages 315, 316 may be located on a side or sides of the distal portion 310 (i.e., between surfaces 311, 312), in addition to or instead of locating the passages on either or both of surfaces 311, 312.

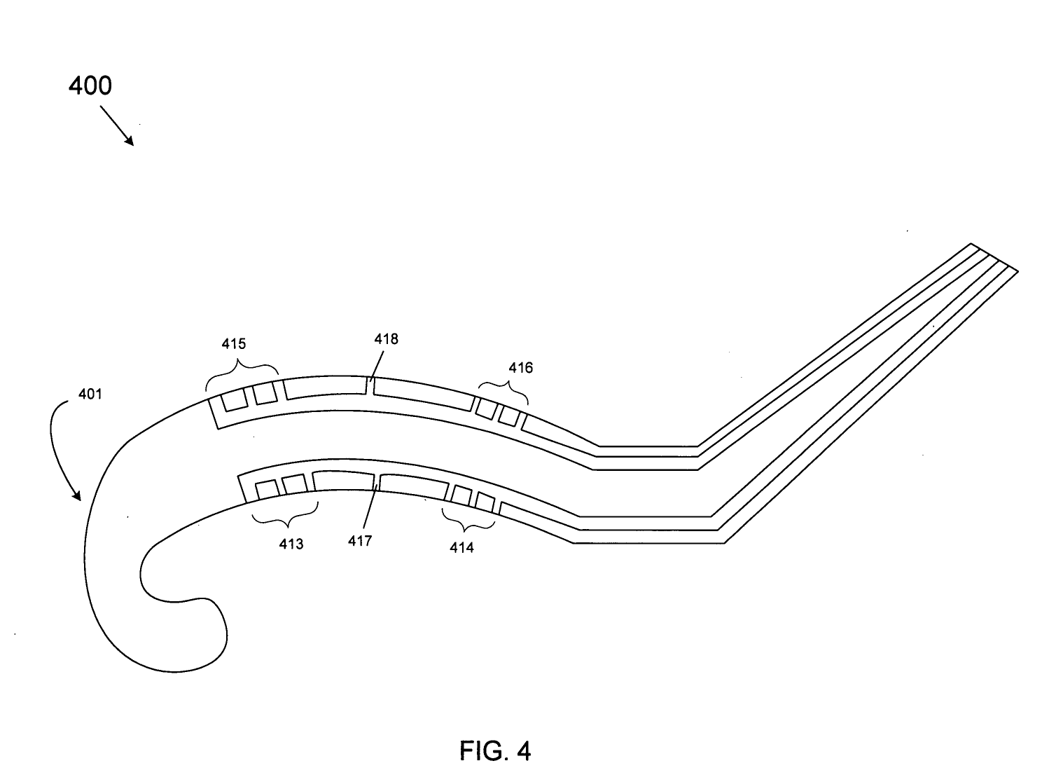

FIG. 4 includes a side view of one embodiment of the invention. The device 400 includes a lip 401 and passages 413, 417, 414, 415, 418, 416. Passages 413, 417, 414 may couple with the donor tissue. Collectively, passages 413, 417, 414 have a concentration of passages towards the outer perimeter of the region that mates with the donor tissue. This concentration may help better hold, for example, the distal edge of the donor tissue to the device as the donor tissue is inserted into the eye. A similar concentration along an outer perimeter may be used for irrigation/aspiration (e.g., fluid addition, fluid flushing, fluid removal) passages 415, 418, 416. The lip 401 may help protect the distal edge of the donor tissue when the donor tissue is inserted through an incision and into the eye. As a result, the magnitude of negative pressure used to couple the donor tissue to the device 400 may be lessened. In one embodiment on the invention, the lip 401 extends approximately 250 μm below the surface of the device where the passages 413, 417, 414 are located.

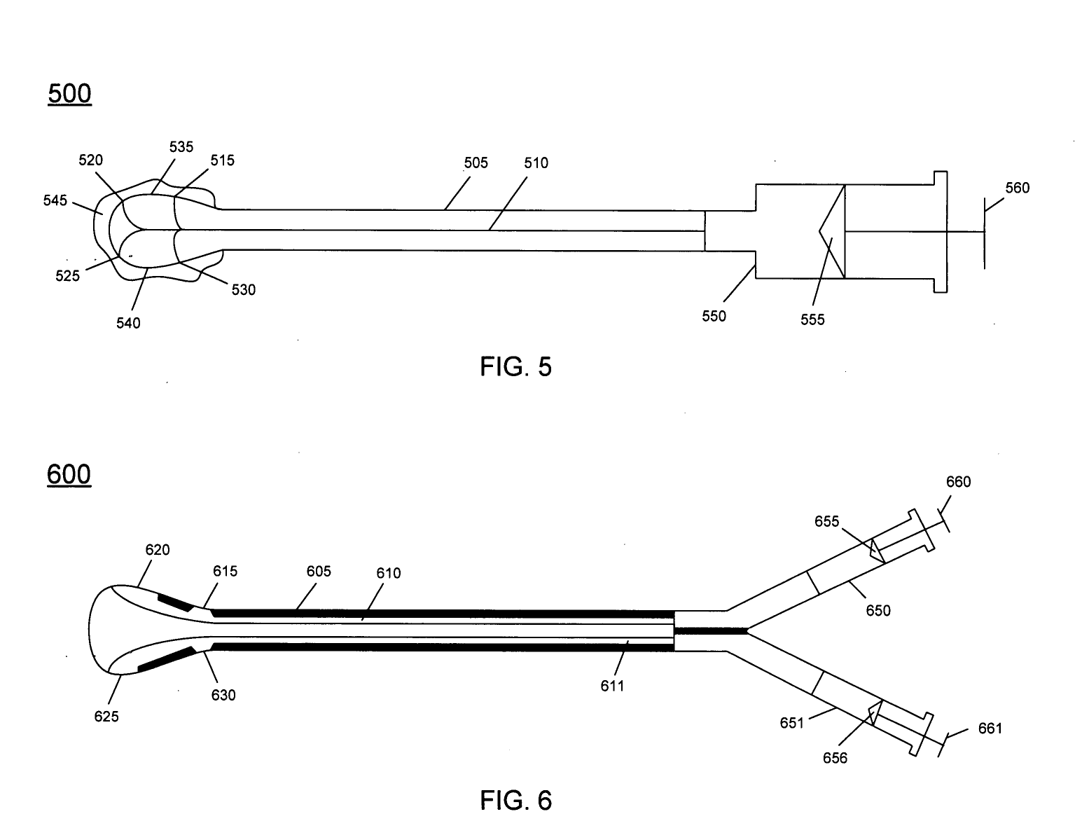

FIG. 5 includes a side sectional view of one embodiment 500 of the invention. A syringe 550 may include a fluid (e.g., air, saline) that can be advanced/withdrawn (positively or negatively) by use of the plunger 555 and handle 560. The fluid may advance, via channel 510 to holes 515, 520, 525, 530. Channel 510 may be included within housing/conduit 505. Holes 515, 520 may be included on a first surface 535 while holes 525, 530 are included on a second surface 540, located substantially opposite first surface 535. Donor tissue 545 may wrap or roll about the first surface 535 and second surface 540. A stromal surface of tissue 545 may directly contact a surface (e.g., 535), however, a different surface (e.g., endothelial surface) may instead directly contact a surface (e.g., 535).

FIG. 6 includes a side sectional view of one embodiment 600 of the invention. A syringe 650 may include a fluid (e.g., air) that can be advanced (positively or negatively) by use of the plunger 655 and handle 660. The fluid may advance, via channel 610, to holes 615, 620. Channel 610 may be included within housing/conduit 605. Another syringe 651 (or any other fluid source) may include a fluid (e.g., saline) that can be advanced (positively or negatively) by use of the plunger 656 and handle 661. The fluid may advance, via channel 611, to holes 625, 630. Channel 611 may be included within housing/conduit 605.

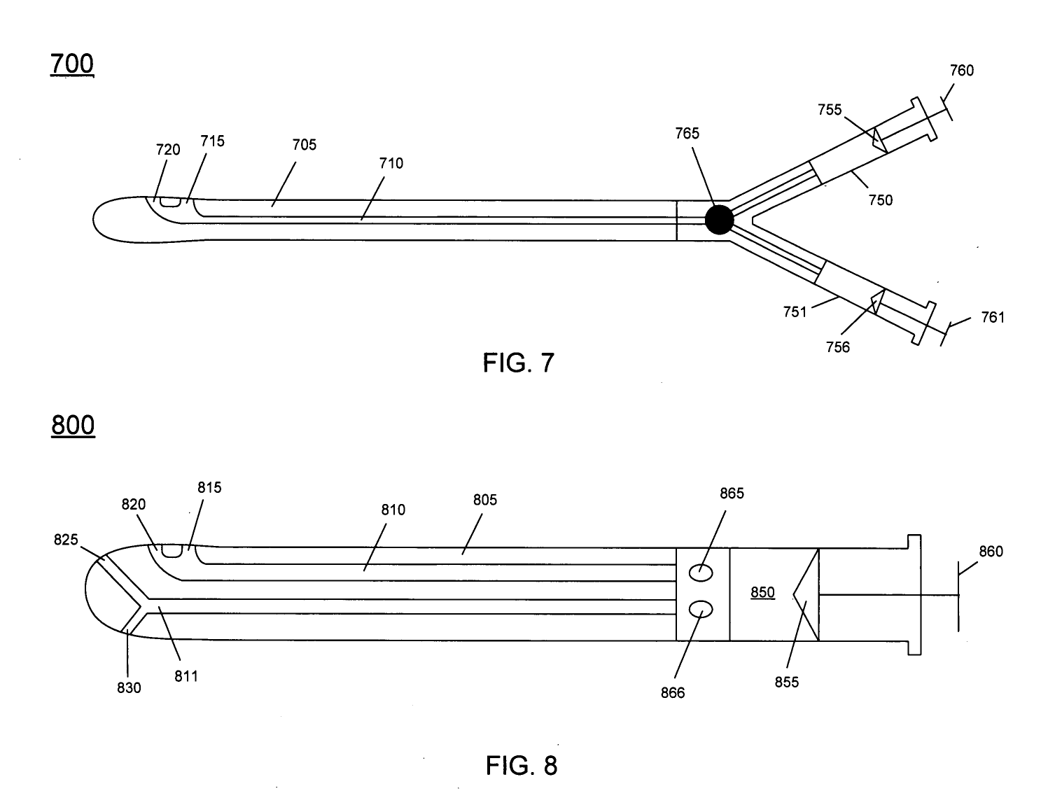

FIG. 7 includes a side sectional view of one embodiment 700 of the invention. A syringe 750 may include a fluid (e.g., air) that can be advanced (positively or negatively) by use of plunger 755 and handle 760. The fluid may advance, via channel 710, to holes 715, 720. Channel 710 may be included within housing/conduit 705. Another syringe 751 (or any other fluid source) may include a fluid (e.g., saline) that can be advanced (positively or negatively) by use of the plunger 756 and handle 761. The fluid may advance, via channel 710, to holes 715, 720. A valve 765 may be configured to select between fluid sources 750, 751.

FIG. 8 includes a side sectional view of one embodiment 800 of the invention. A syringe 850 may include a fluid (e.g., air) that can be advanced (positively or negatively) by use of plunger 855 and handle 860. The fluid may advance, via channel 810, to holes 815, 820. Channel 810 may be included within housing/conduit 805. Another syringe can be substituted for syringe 850 (or any other fluid source) with the additional syringe including another fluid (e.g., saline) that can be advanced (positively or negatively) as described above. Of course a user may simple exchange, for example only, air for saline in syringe 850. Valves 865, 866 may be configured to respectively control fluid flow in channels 810, 811.

In an alternative embodiment, the device and method is not limited to endothelial keratoplasty but is applicable to other procedures such as, for example, standard corneal transplant and other tissue (e.g., non-ophthalmic tissue) transplants.

Thus, an ophthalmic surgical apparatus may include a distal portion having a first passage. The distal portion can be coupled to a donor ophthalmic tissue and inserted into an eye via an incision (e.g., 5 mm in maximum breadth). The apparatus may also include a proximal portion having a second passage which couples an air pressure source to the first passage. The first and second passages may be continuous and indistinct from one another but may also be, for example, separably coupled to one another with or without intervening passages in between the first and second passages. The distal portion may include a first surface and a second surface with the first surface including a first hole and a second hole. The holes may couple the first passage and air pressure source (e.g., vacuum) to the donor ophthalmic tissue. These holes, and/or additional holes, may also supply a liquid (e.g., irrigation solution) to the eye. The holes and passages may be continuous and indistinct from one another but may also be, for example, separably coupled to one another with or without intervening passages or conduits in between the hole or void and passage. Additional passages may be included in the device. For example, a third passage may be coupled to a third hole and a liquid source. The third passage may couple to the liquid source via the second passage or, for example, via a passage other than the second passage. A third hole can be included on a second surface located substantially opposite the first surface. The first surface, for example only, may include a concave surface to couple to stromal tissue. The holes, possibly used for (as an example only) suction and/or irrigation, may be concentrated more heavily in one region (e.g., perimeter) of a particular surface or surfaces of the device. A protective protrusion (e.g., protective lip or shield) may shield sensitive tissue (e.g., endothelial cells) during advancement of the distal portion of the device into the incision and within the eye. More than one such lip can located on the device. For example, another lip can be added on the proximal side of the distal portion of the device to help protect tissue as the tissue is maneuvered in the eye and possibly extracted from the eye. The device may allow for a sutureless coupling of a donor endothelial lining to the patient's own tissue in a DSAEK procedure. This sutureless coupling of tissues stands in contrast with traditional techniques like the more aggressive “open sky” complete corneal transplants, which require suturing of donor tissue to patient tissue.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. A method comprising:
applying a negative pressure to holes, included in a first surface of an endothelial keratoplasty device, via a conduit that couples the holes to a negative pressure source;
coupling a stromal surface of a donor ophthalmic endothelial tissue to the holes using the negative pressure;
inserting the first surface and donor ophthalmic endothelial tissue into a patient's eye;
discontinuing the application of negative pressure to the holes decoupling the holes from the stromal surface of the donor ophthalmic endothelial tissue;
coupling the stromal surface of the donor ophthalmic endothelial tissue to the patient's corneal stroma; and
removing the endothelial keratoplasty device from the eye.

2. The method of claim 1, further comprising injecting a fluid into the patient's eye via one or more of the holes to decouple the holes from the stromal surface of the donor ophthalmic endothelial tissue.

3. The method of claim 1, further comprising directly connecting the stromal surface of the donor ophthalmic endothelial tissue to the holes using the negative pressure.

4. The method of claim 1, wherein the first surface directly contacts at least 18 mm² of the stromal surface of the donor ophthalmic endothelial tissue.

5. The method of claim 1, wherein the stromal surface of the donor ophthalmic endothelial tissue includes an outermost breadth of less than 10 mm and a central region that includes a diameter of less than 5 mm, and further wherein the there are no creases impinged upon the central region of the donor ophthalmic endothelial tissue during the method.

6. The method of claim 1, further comprising:
applying the negative pressure to additional holes included in a second surface of the endothelial keratoplasty device, the second surface located substantially opposite the first surface; and
coupling the stromal surface of the donor ophthalmic endothelial tissue to the additional holes using the negative pressure.

7. The method of claim 1, further comprising maintaining the shape of the anterior chamber of the patient's eye via fluid produced from the endothelial keratoplasty device.

8. The method of claim 1, further comprising inserting the first surface and donor ophthalmic endothelial tissue into a patient's eye via an incision, the incision including a maximum breadth of less than 6 mm.

9. An ophthalmic surgical apparatus comprising:
a distal portion having a first passage, the distal portion to insert into an eye and to couple to a donor ophthalmic tissue; and
a proximal portion having a second passage, the second passage to couple an air pressure source to the first passage;
wherein the distal portion includes a first surface and a second surface, the first surface including a first hole and a second hole each of which is to operatively couple the first passage and air pressure source to the donor ophthalmic tissue.

10. The apparatus of claim 9, wherein the second passage is to further couple a liquid source to the first passage.

11. The apparatus of claim 9, wherein the first hole and second hole are to couple a negative air pressure from the air pressure source to the donor ophthalmic tissue.

12. The apparatus of claim 9, wherein the distal portion includes a third passage coupled to a third hole, the third passage to couple to a liquid source.

13. The apparatus of claim 12, wherein the third passage is to couple to the liquid source via the second passage.

14. The apparatus of claim 12, wherein the third passage is to couple to the liquid source via a fourth passage, the fourth passage included in the proximal portion.

15. The apparatus of claim 12, wherein the third hole is included in the second surface, the second surface located substantially opposite the first surface.

16. The apparatus of claim 9, wherein the first surface includes a concave surface.

17. The apparatus of claim 9, further comprising a first plurality of holes including the first and second holes, wherein the first plurality of holes are more densely populated along a perimeter of the distal portion than along a central region of the distal portion.

18. The apparatus of claim 9, wherein the distal portion includes a first protective protrusion extending away from the distal portion to shield, during advancement of the distal portion in the distal direction, the donor ophthalmic tissue coupled to the distal portion.

19. An apparatus comprising:
an endothelial keratoplasty device including a distal portion having a first passage, the distal portion to insert into an eye and to couple to a donor ophthalmic tissue; and
a proximal portion, included in the endothelial keratoplasty device, having a second passage, the proximal portion including a second passage to couple to a fluid source;
wherein the distal portion comprises a first surface including a first plurality of voids to couple the first passage and fluid source to the donor ophthalmic tissue.

20. The apparatus of claim 19, wherein the first plurality of voids are to couple a negative pressure from the fluid source to the donor ophthalmic tissue and a second plurality of voids, included in the first surface, are to supply a liquid from another fluid source to the eye.
